(19) United States
(12) Patent Application Publication
(10) Pub. No.: US 2024/0180970 A1
(43) Pub. Date: Jun. 6, 2024

(54) METHODS OF TREATMENT FOR FAILURE TO THRIVE

(71) Applicant: PROLACTA BIOSCIENCE, INC., Duarte, CA (US)

(72) Inventor: Melinda J. ELLIOTT, Duarte, CA (US)

(21) Appl. No.: 18/552,081

(22) PCT Filed: Mar. 25, 2022
(86) PCT No.: PCT/US2022/021860

§ 371 Date: Sep. 22, 2023

Related U.S. Application Data
(60) Provisional application No. 63/166,400, filed on Mar. 26, 2021.

(51) Int. Cl. A61K 35/20; A23L 33/00; A61K 9/00; A61P 3/02

(52) U.S. Cl. CPC A61K 35/20; A23L 33/40; A61K 9/0053; A61P 3/02

(57) ABSTRACT

Provided herein are compositions and methods useful for improving or increasing weight gain or growth velocity in a subject, e.g., an infant having or suspected of having failure to thrive. In some aspects, the methods are or include administering a human milk cream composition to increase or improve growth velocity or weight gain.

### CROSS-REFERENCE TO RELATED APPLICATIONS

This application claims priority from U.S. provisional application No. 63/166,400 filed Mar. 26, 2022, entitled “METHODS FOR TREATMENT FOR FAILURE TO THRIVE”, the contents of which are incorporated by reference in its entirety.

### FIELD OF THE INVENTION

Provided herein are compositions and methods useful for improving or increasing weight gain or growth velocity in a subject in need thereof, e.g., an infant having or suspected of having failure to thrive. In some aspects, the methods are or include feeding or administering a human milk cream composition to increase or improve growth velocity or weight gain.

### BACKGROUND OF THE INVENTION

Failure to thrive, also known as weight faltering, is a common term used to describe lack of adequate weight gain in pediatric patients. Failure to thrive may be defined as a weight for age that falls below the tenth or fifth percentile on multiple occasions or weight deceleration that crosses two major percentile lines on a growth chart. Failure to thrive can result in developmental delays and other long-term effects for the developing child. In the United States, it has been estimated that as many as five to ten percent of children in primary care settings have failure to thrive. While it may result from various underlying physical or psychosocial causes, failure to thrive generally involves a lack of necessary calories for adequate growth. This could be from not taking in enough calories, losing too many calories, or increased caloric demand.

Treatment plans for breastfed babies or infants often involve supplemental feedings with cow milk or plant-based formula or discontinuing breastfeeding and replacing with cow milk or plant-based formula feedings. Even when breastfeeding is continued, the supplemental formula feedings are often disruptive for breastfeeding. While such strategies may result in weight gain, they are less than ideal given the long-term benefits of breastfeeding. What is needed in the art are compositions and methods that for improving growth in infants with failure to thrive that are compatible with breastfeeding.

### SUMMARY OF THE INVENTION

Provided herein are methods for treating, ameliorating, or remedying failure to thrive and/or improving or increasing growth velocity or weight gain in a subject, e.g., an infant, having or suspected of having failure to thrive. In some embodiments, the methods are or include feeding or administering a human milk cream composition to the subject in need thereof. In certain embodiments, the human milk cream composition is or includes 20% to 30% human milk fat, e.g., weight/weight (w/w) or weight/volume (w/v) and/or 2.0 kcal/mL to 3.0 kcal/mL.

Provided herein is a method of treating failure to thrive in a subject, the method comprising administering to a subject in need thereof a human milk cream composition, wherein the human milk cream composition comprises from 20% to 30% human milk fat, e.g., w/w or w/v, and from 2.0 kcal/mL to 3.0 kcal/mL. In some embodiments, the human milk cream composition is free or essentially free of macronutrients of a non-human milk origin.

In some embodiments, the human milk cream composition is administered orally. In particular embodiments, the composition is administered enterally. In some embodiments, the human milk cream composition is not mixed or diluted with one or more compositions prior to administering. In some embodiments, the human milk cream composition is mixed with human milk prior to administering. In some embodiments, the human milk cream composition is administered to the subject in a dose of from 1 mL to 20 mL. In some embodiments, the subject is administered at least one dose of the human milk cream composition per day. In some embodiments, the subject is administered the human milk cream composition from 5 mL to 100 mL per day, optionally 5 mL to 50 mL per day. In some embodiments, the human milk cream composition is administered to the subject in an amount sufficient for the subject to achieve a growth rate of at least 20 g/day. In some embodiments, the human milk cream composition is administered to the subject in an amount sufficient for the subject to achieve a growth rate of 20 g/day to 30 g/day. In some embodiments, the subject is less than two years old. In certain embodiments, the subject is less than one year old. In some embodiments, the subject is breastfed and/or receives an exclusive human milk diet. In some embodiments, the subject is a term infant, a late preterm infant, and/or was at least 34 weeks gestation at birth.

In some embodiments, the failure to thrive is associated with or suspected of being associated with inadequate calorie intake, inadequate nutrient absorption, or increased metabolism. In some embodiments, the failure to thrive is associated with or suspected of being associated with inadequate calorie intake, wherein the inadequate calorie intake is associated with one or more of gastroesophageal reflux, inadequate breast milk supply or ineffective latching, mechanical feeding difficulties, poor oral neuromotor coordination, toxin-induced or infectious gastrointestinal upset. In certain embodiments, the failure to thrive is associated with a food intolerance, e.g., to a non-human milk such as cow's milk or soy milk. In particular embodiments, the failure to thrive is associated with a food allergy, e.g., to a non-human milk such as cow's milk or soy milk. In some embodiments, the failure to thrive is associated with or suspected of being associated with inadequate nutrient absorption, wherein the inadequate nutrient absorption is associated with one or more of anemia or iron deficiency, biliary atresia, celiac disease, or chronic gastrointestinal conditions. In some embodiments, the failure to thrive is associated with or suspected of being associated with increased metabolism, wherein the increased metabolism is associated with a chronic infection, chronic lung disease of prematurity, congenital or acquired cardiovascular or heart disease, hyperthyroidism, inflammatory conditions, malignancy, congenital adrenal overactivity, hypertension, a hormone secreting tumor (e.g., a non-malignant hormone secreting tumor such as pheochromocytoma), or renal dysfunction.

In some embodiments, the subject has or is suspected of having congenital anomalies, developmental delay, gastroesophageal reflux, low birth weight, or poor oral health. In some embodiments, the subject has (i) weight below the 10th percentile for sex and corrected age: (ii) weight for length below the 10th percentile: (iii) body mass index for age below the 10th percentile: and/or (iv) a decrease in growth velocity, whereby the subject's weight for age or weight for length/height has fallen by two major percentiles since birth, wherein the major percentiles are 95%, 90%, 75%, 50%, 25%, 10%, and 5%.

In some embodiments, the human milk cream composition has been treated with a treatment to reduce bioburden. In some embodiments, the treatment to reduce bioburden comprises pasteurization. In some embodiments, the human milk cream composition is pasteurized. In some embodiments, the human milk cream composition comprises cream from human milk of multiple donors. In some embodiments, the cream is from human milk of at least 5, 10, 25, 50, or 100 individual donors.

In some embodiments, the human milk cream composition comprises from 24% to 26% human milk fat, e.g., w/v, and from 2.25 kcal/mL to 2.75 kcal/mL. In some embodiments, the human milk cream composition comprises about 25% human milk fat, e.g., w/v; and 2.5kcal/mL to 2.7 kcal/mL. In some embodiments, provided herein is a use of a human milk cream composition for treating failure to thrive in a subject in need thereof, wherein the human milk cream composition comprises from 20% to 30% human milk fat, e.g., w/v, and from 2.0 to 3.0 kcal/mL.

In some embodiments, provided herein is a human milk cream composition for use in the manufacture of a medicament for the treatment of failure to thrive in a subject in need thereof, wherein the human milk cream composition comprises from 20% to 30% human milk fat, e.g., w/v.

Also provided herein is a method for inducing weight gain in a subject in need thereof, wherein the subject's weight for age or weight for length/height has fallen by two major percentiles since birth, the method comprising orally or enterally administering to the subject in need thereof a human milk cream composition comprising from 20% and 30% human milk fat, e.g., w/v, and from 2.0 and 3.0 kcal/mL: wherein the subject is less than 2 years old, was at least 34 weeks gestational age at birth, and is breastfed or exclusively breastfed, wherein the human milk cream composition is administered at least once daily for a period of time sufficient for the subject's weight for age or weight for length/height to increase by two major percentiles, wherein the major percentiles are 95%, 90%, 75%, 50%, 25%, 10%, and 5%.

In addition, provided herein is a method for inducing weight gain in a subject in need thereof, wherein the subject's weight for age, weight for length/height, or body mass index for age is below the tenth percentile, the method comprising orally or enterally administering to the subject in need thereof a human milk cream composition comprising from 20% and 30% human milk fat, e.g., w/v, and from 2.0 and 3.0 kcal/mL: wherein the subject is less than 2 years old, was at least 34 weeks gestational age at birth, and is breastfed or exclusively breastfed, wherein the human milk cream composition is administered at least once daily for a period of time sufficient for the subject's weight for age, weight for length/height, or body mass index for age to increase to at least the tenth percentile.

In some embodiments, from 25 kcal to 60 kcal of the human milk cream composition is administered daily. In certain embodiments, from 10 kcal to 100 kcal of the human milk cream composition is administered daily. In some embodiments, the human milk cream composition comprises about 25% human milk fat, e.g., w/v, and 2.5 kcal/mL to 2.7 kcal/mL.

### DETAILED DESCRIPTION

Provided herein are methods for treating, ameliorating, or remedying failure to thrive and/or improving or increasing growth velocity or weight gain in a subject, e.g., an infant, having or suspected of having failure to thrive. In some embodiments, the methods are or include feeding or administering a human milk cream composition to the subject in need thereof. In certain embodiments, the human milk cream composition is or includes 20% to 30% human milk fat, e.g., weight/volume (w/v) or weight/weight (w/w).

Failure to thrive, sometimes referred to as weight faltering, can result in developmental delays and other long-term effects for the developing child. While failure to thrive may result from various underlying physical or psychosocial causes, failure to thrive generally involves a lack of necessary calories for adequate growth. This could be from not taking in enough calories, losing too many calories, or increased caloric demand. Current strategies for improving growth and weight gain in these infants often involve discontinuation of breastfeeding for formula feeding or supplemental feedings that may be disruptive to breastfeeding. The benefits of breastfeeding over formula feeding are well documented and include short and long term developmental and health benefits for the infant. Thus, while improving growth and weight gain is critically important for infants with failure to thrive, what is needed in the art are new treatments for failure to thrive that are compatible with and non-disruptive to breastfeeding and/or maintaining an exclusive human milk diet.

The provided methods and compositions address these needs. A main advantage of the provided human milk cream compositions is that they are not disruptive to breastfeeding. The human milk cream composition exclusively contains only human milk proteins, fats, and carbohydrates and do not disrupt the infant's preference for breastmilk or ability to breastfeed. In some embodiments, the human milk cream composition may be administered orally, such as with a needleless syringe, to the infant prior to a breastfeeding. The infant may be placed on the mother's breast for breastfeeding immediately after the human milk cream composition is administered. Alternatively, the human milk cream composition may be mixed with expressed breastmilk or donor milk prior to feeding.

Breastmilk is considered to be the optimal nutrition for newborns and infants and provides benefits over formula or other alternative nutrition. The World Health Organization (WHO) recommends mothers worldwide to exclusively breastfeed infants for the child's first six months to achieve optimal growth, development, and health. While traditional interventions for failure to thrive rely on formulas or supplements derived from cow milk or plant sources, the provided compositions and methods allow for the treatment of failure to thrive while maintaining an exclusive human milk diet for the infant. Furthermore, in some aspects, feedings with the human milk cream composition does not disrupt or alter the infant's intestinal microbiome, e.g., as compared to the intestinal microbiome of an exclusively breastfed infant, owing at least in part to the absence of non-human proteins, fats, and carbohydrates. Thus, in some aspects, an advantage of the provided methods and compositions is the ability to treat failure to thrive while maintaining the infant on an exclusive human milk diet.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications, and other publications that are herein incorporated by reference, the definition set forth herein prevails over the definition that is incorporated herein by reference.

The section heading used herein are for organizational purposes only and are not to be construed as limiting the subject matter described.

### I. Methods for Treating Failure to Thrive

Provided herein are methods for treating, ameliorating, remedying, or improving failure to thrive that are or include feeding or administering a human milk cream composition to a subject in need thereof. In some embodiments, the human milk cream composition is administered, e.g., orally, to an infant that has, is suspected of having, or is at risk for failure to thrive. In some embodiments, the human milk cream composition contains human milk fat, human milk protein, and human milk carbohydrates (e.g., fats, proteins, and carbohydrates derived from, obtained from, or found in human milk). In certain embodiments, the human milk cream composition is free of or essentially free of non-human fats, proteins, or carbohydrates. In particular embodiments, the human milk cream composition is free of or essentially free of non-human milk proteins.

In certain embodiments, the subject is under two years of age. In particular embodiments, the subject is under 1 year of age. In some embodiments, the subject is under 6 months of age. In particular embodiments, the subject is an infant. In some embodiments, the subject was born at full term or at late preterm. In certain embodiments, the subject was born at a gestational age of at least 32, 33, 34, 35, or 36 weeks. In particular embodiments, the subject was born at a gestational age of at least 34 weeks. In some embodiments, the subject is breastfed. In particular embodiments, the subject is exclusively breastfed. In certain embodiments, the infant is fed an exclusive human milk diet. In particular embodiments, the subject is a term infant or an infant born at full term. In some embodiments, the subject was born at least at 37 weeks gestation. In certain embodiments, the subject was born at from at least 39 weeks gestation to 40 weeks, 6 days gestation. In some embodiments, the subject is a late term infant or an infant born at late term. In particular embodiments, the subject was born at from at least 40 weeks gestation, e.g., between 40 weeks gestation and 41 weeks, 6 days gestation. In some embodiments, the subject is a post-term infant. In particular embodiments, the subject was born at a gestational age of at least 42 weeks. In certain embodiments, the subject is an early-term infant. In some embodiments, the subject was born at from 37 weeks to 38 weeks, 6 days gestation.

In some embodiments, the provided methods are or include feeding or administering a human milk cream composition, e.g., a human milk cream composition described herein such as in Section II, to a subject, e.g., an infant, that has or is suspected of having failure to thrive. In some embodiments, the failure to thrive is or includes: weight below the fifth percentile for sex and corrected age: weight for length below the fifth percentile: body mass index for age below the fifth percentile: and/or a decrease in growth velocity resulting in a drop of at least two major percentiles for weight for age or weight for length/height. In certain embodiments, the failure to thrive is or includes: weight below the tenth percentile for sex and corrected age: weight for length below the tenth percentile: and/or a body mass index for age below the tenth percentile. Particular embodiments contemplate that a subject's, e.g., an infant's, percentile for sex and corrected age, weight for length or height, or BMI for age can be determined as a matter of routine, such as by consulting the World Health Organization's (WHO's) charts length/height-for-age, weight-for-age, weight-for-length, weight-for-height, and body mass index-for-age (see, e.g., WHO Multicentre Growth Reference Study Group. WHO child growth standards: length/height-for-age, weight-for-age, weight-for-length, weight-for-height and body mass index-for-age: methods and development. Geneva: World Health Organization: 2006) or those provided by the Center of Disease Control (CDC: see, e.g., Kuczmarski R J, Ogden C L, Guo S S, Grummer-Strawn L M, Flegal K M, Mei Z, et al. 2000 CDC Growth Charts for the United States: methods and development. Vital & Health Statistics Series. 2002: 11:1-190), each hereby incorporated by reference in their entirety.

In particular embodiments, the provided methods are or include feeding or administering a human milk cream composition, e.g., a human milk cream composition described herein such as in Section II, to a subject, e.g., an infant, in need of improved or increased growth velocity and/or weight gain. In some embodiments, the subject is an infant who is underweight and/or has or displays slow or no growth velocity or weight gain. In particular embodiments, the subject has low weight for age and sex, e.g., weight that is less than the tenth or fifth percentile for sex and corrected age such as according to WHO or CDC growth charts. In certain embodiments, the subject is an infant with low weight for length, e.g., below the tenth or fifth percentile for weight for length such as according to WHO or CDC growth charts. In some embodiments, the subject is an infant with a low body mass index (BMI) for age, e.g., a BMI below the tenth or fifth percentile such as according to WHO or CDC growth charts. In particular embodiments, the subject is experiencing or has experienced a decrease in growth velocity, such as where the subject's weight for age or weight for length/height has fallen by two major percentiles e.g., since birth, such as according to WHO or CDC growth charts.

In some embodiments, the subject requires, needs, or would benefit from increased growth velocity and/or weight gain. In certain embodiments, the subject receives or is suspected of receiving an inadequate calorie or nutrient intake. In particular embodiments, the subject experiences or is suspected of experiencing inadequate nutrient absorption. In some embodiments, the subject has or is suspected of having an increased metabolism and/or increased metabolic demands.

In particular embodiments, the subject receives or is suspected of receiving an inadequate calorie intake. In certain embodiments, inadequate calorie or nutrient intake may be caused by, related to, and/or associated with one or more of gastroesophageal reflux, inadequate breast milk supply or ineffective latching, incorrect formula preparation, mechanical feeding difficulties (e.g., cleft lip or palate), neglect or abuse, poor feeding habits, poor oral neuromotor coordination, toxin-induced gastrointestinal upset (e.g., elevated lead levels leading to anorexia, constipation, or abdominal pain), or infectious gastrointestinal upset.

In particular embodiments, the subject experiences or is suspected of experiencing inadequate nutrient absorption. In particular embodiments, inadequate nutrient absorption may be caused by, related to, and/or associated with one or more of anemia or iron deficiency, biliary atresia, celiac disease, chronic gastrointestinal conditions (e.g., irritable bowel syndrome), gastrointestinal infections, cystic fibrosis, inborn errors of metabolism, milk protein allergy, or pancreatic cholestatic conditions.

In certain embodiments, the subject has or is suspected of having an increased metabolism and/or increased metabolic demands. In some embodiments, increased metabolism and/or increased metabolic demands may be caused by, related to, and/or associated with one or more of a chronic infection, (e.g., HIV, AIDS, or tuberculosis), chronic lung disease of prematurity, congenital or acquired heart or cardiovascular disease, hyperthyroidism, inflammatory conditions (e.g., asthma or inflammatory bowel disease), malignancy, tumor, cancer, renal disease or dysfunction, lung malformation, vascular malformation, congenital adrenal overactivity, hypertension, or non-malignant, hormone-secreting tumors (e.g., pheochromocytoma).

In particular embodiments, the subject has or is suspected of having a medical condition that may be associated with decreased growth or weight gain, low or no growth or weight gain, and/or failure to thrive. In some embodiments, the medical condition is one or more of a congenital anomaly (e.g., cerebral palsy, autism, or trisomy 21), developmental delay, gastroesophageal reflux, low birth weight (e.g., under 2500 g or 5 lb. 8 oz.), poor oral health, or tongue-tie.

In certain embodiments, the subject has or is suspected of having poor oral health. In some embodiments, the poor oral health is, includes, or is characterized by dental caries, poor tongue coating, plaque, and/or calculus. In particular embodiments, the poor oral health is due to and/or accompanies a congenital or genetic disorder. In some embodiments, the poor oral health accompanies a congenital heart or cardiovascular defect. In some embodiments, the subject has or is suspected of having poor oral health that causes difficulty or discomfort with feeding and/or otherwise interferes with the ability to feed, initiate feeding behavior, and/or complete full feeds.

In some embodiments, the subject has or is suspected of experiencing a psychosocial or family issue that may be associated with decreased growth or weight gain, low or no growth or weight gain, and/or failure to thrive. In certain embodiments, the psychosocial or family issue is one or more of disordered feeding techniques, family stressors, parental or family history of domestic abuse or violence or intimate partner abuse or violence (perpetrator or victim), poor parenting skills, postpartum depression, poverty, social isolation of a caretaker, substance abuse, or unusual health and nutritional beliefs (such as restricted diets).

In particular embodiments, the subject is an infant who will undergo or who has undergone surgery for a congenital defect or condition. Particular embodiments contemplate that encouraging robust growth and/or maintaining or increasing the growth trajectory in infants, e.g., breastfed infants, with conditions that will or may require surgery will increase clinical outcomes and recovery following surgery. Furthermore, in certain embodiments, infants recovering from surgery may have increased metabolic demands, e.g., as compared to infants of similar age, weight, and/or length/height that did not require surgery and/or did not have the congenital defect or condition. In particular embodiments, the human milk cream composition is fed or administered to a subject prior to a surgery. In some embodiments, the human milk cream composition is fed or administered to the subject following a surgery.

In some embodiments, the surgery is a corrective surgery to treat, address, correct, or ameliorate a congenital heart defect. In particular embodiments, the surgery is or includes patent ductus arteriosus (PDA) ligation, coarctation of the aorta repair, atrial septal defect (ASD) repair, ventricular septal defect (VSD) repair, tetralogy of Fallot repair, transposition of the great vessels repair, truncus arteriosus repair, tricuspid atresia repair, total anomalous pulmonary venous return (TAPVR) correction, or hypoplastic left heart repair.

In some embodiments, the surgery is a surgery (e.g., corrective surgery) to treat, address, correct, or ameliorate a congenital gastrointestinal disorder (CGD). In some embodiments, the CGD is or includes gastroschisis, omphalocele, malrotation, diaphragmatic hernia, atresia, esophageal atresia, atresia in the jejunoileal region, atresia in the duodenum, esophageal obstruction, gastric obstruction, duodenal obstruction, jejunal obstruction, intestinal duplication, colonic atresia, congenital tumor removal, gut cell tumor removal, germ cell tumor removal, surgery for biliary atresia or Kasai procedure, or Hirschsprung disease.

In some embodiments, the surgery is a surgery to treat, remedy, ameliorate, correct, or address one or more of a congenital pulmonary condition, congenital diaphragmatic hernia, congenital pulmonary adenomatoid malformation, pulmonary sequestration. In certain embodiments, the surgery is a surgery to treat, remedy, ameliorate, or remove a cancer. In some embodiments, the surgery is a surgery to treat, remedy, ameliorate, or remove a teratoma or a germ cell tumor. In some aspects, teratomas may be located anywhere within the body, but are typically associated, e.g., in infant subjects born at full term, with the sacral area or in the chest. In certain embodiments, the surgery is a surgery to treat, remedy, ameliorate, correct, or address severe reflux. In some embodiments, the surgery is a fundoplication for severe reflex.

In certain embodiments, the human milk cream composition is orally administered to the subject. In particular embodiments, the human milk cream composition is administered to the subject without first diluting or mixing the human milk cream composition with any other composition, such as with the infant's mother's own milk (also referred to herein as mother's own milk or MOM), donor human milk, or infant formula. In certain embodiments, the human milk cream composition is administered orally to the subject, e.g., with a feeding syringe, prior to breastfeeding. In certain embodiments, the infant is breastfed immediately after an oral feeding of the human milk cream composition.

In some embodiments, the human milk cream composition is administered enterally such as through a feeding tube, e.g., a nasogastric tube, an orogastric tube, a gastrostomy tube, a gastro-jejunostomy tube, or a jejunostomy tube. In certain embodiments, the human milk cream composition is administered to the subject enterally through a nasogastric or an orogastric tube. In some embodiments, the human milk cream composition is enterally administered to the subject without first diluting or mixing the human milk cream composition with any other composition, such as with human milk, e.g., MOM or donor milk, or infant formula.

In some embodiments, the human milk cream composition is diluted or mixed with the infant's mother's own milk (also referred to herein as mother's own milk or MOM) or donor human milk prior to feeding or administering. In some embodiments, the human milk cream composition is diluted or mixed with an infant formula prior to feeding or administering. In some embodiments, the infant formula is a human milk derived ready to feed formulation, e.g., free or essentially free from non-human protein, fats, lipids, or carbohydrates.

In particular embodiments, the human milk cream composition is administered, e.g., orally, in a manner sufficient to increase growth velocity. In some embodiments, the human milk cream composition is administered, e.g., orally, in a manner sufficient to increase the rate of weight gain. In certain embodiments, the human milk cream composition is administered, e.g., orally, in a manner sufficient to increase the subject's weight for age or weight for length/height by at least one, five, or ten percentiles. In some embodiments, the human milk cream composition is administered, e.g., orally, in a manner sufficient to increase the subject's weight for age or weight for length/height by at least one major percentile (e.g., 95%, 90%, 75%, 50%, 25%, 10%, and 5% percentiles). In particular embodiments, the human milk cream composition is administered, e.g., orally, in a manner sufficient to increase the subject's weight for age or weight for length/height by at least two major percentiles. In certain embodiments, the human milk cream composition is administered, e.g., orally, in a manner sufficient to bring one or more of the subject's weight, weight for length, or BMI for age above the fifth percentile.

In some embodiments, the human milk cream composition is fed or administered to a subject, e.g., an infant, to increase or improve weight gain or growth velocity. In certain embodiments, the human milk cream composition is administered, e.g., orally, to the subject to obtain, achieve, or maintain, a growth velocity or rate of weight gain of at least 1 g/day, 2 g/day, 3 g/day, 4 g/day, 5 g/day, 6 g/day, 7 g/day, 8 g/day, 9 g/day, 10 g/day, 12 g/day, 14 g/day, 15 g/day, 16 g/day, 18 g/day, 20 g/day, 25 g/day, 30 g/day, 35 g/day, 40 g/day, 45 g/day, or 50 g/day. In some embodiments, the human milk cream composition is administered, e.g., orally, to the subject to obtain, achieve, or maintain a growth velocity or rate of weight gain of at least 10 g/day. In particular embodiments, the human milk cream composition is administered, e.g., orally, to the subject to obtain, achieve, or maintain a growth velocity or rate of weight gain of at least 20 g/day. In some embodiments, the human milk cream composition is administered, e.g., orally, to the subject to obtain, achieve, or maintain a growth velocity or rate of weight gain of 20 g/day to 30 g/day. In particular embodiments, the human milk cream composition is administered, e.g., orally, to the subject to obtain, achieve, or maintain a growth velocity or rate of weight gain of more than 30 g/day.

In some embodiments, the human milk cream composition is administered, e.g., orally, to the subject at least once a day over a period of time lasting at least 1 day, 2 days, 3 days, 4 days, 5 days, 6 days, 7 days, 14 days, 21 days, 2 weeks, 3 weeks, 4 weeks, 5 weeks, 6 weeks, 7 weeks, 8 weeks, 9 weeks, 10 weeks, 11 weeks, 12 weeks, 2 months, 3 months, or 6 months. In certain embodiments, a dose, e.g., an oral dose, of the human milk cream composition is administered daily to the subject for at least 1 day, 2 days, 3 days, 4 days, 5 days, 6 days, 7 days, 14 days, 21 days, 2 weeks, 3 weeks, 4 weeks, 5 weeks, 6 weeks, 7 weeks, 8 weeks, 9 weeks, 10 weeks, 11 weeks, 12 weeks, 2 months, 3 months, or 6 months.

In particular embodiments, the human milk cream composition is administered, e.g., orally, to the subject at least once a day to the subject until the subject's weight for age or weight for length/height increases by at least one, five, or ten percentiles. In some embodiments, the human milk cream composition is administered, e.g., orally, to the subject at least once a day to the subject until the subject's weight for age or weight for length/height increases by at least one major percentile. In certain embodiments, the human milk cream composition is administered, e.g., orally, to the subject at least once a day to the subject until the subject's weight for age or weight for length/height increases by at least two major percentiles. In particular embodiments, the human milk cream composition is administered, e.g., orally, to the subject at least once a day until the subject's weight, weight for length, or BMI for age increases above the fifth percentile. In some embodiments, the human milk cream composition is administered, e.g., orally, to the subject at least once a day until the subject's weight, weight for length, or BMI for age increases above the tenth percentile.

In particular embodiments, the subject is administered about or at least 1 kcal, 2 kcal, 2.5 kcal, 3 kcal, 4 kcal, 5 kcal, 6 kcal, 7 kcal, 7.5 kcal, 8 kcal, 9 kcal, 10 kcal, 12.5 kcal, 15 kcal, 17.5 kcal, 20 kcal, 25 kcal, 30 kcal, 35 kcal, 40 kcal, 45 kcal, 50 kcal, 55 kcal, 60 kcal, 62.5 kcal, 65 kcal, 70 kcal, 75 kcal, 80 kcal, 85 kcal, 90 kcal, 95 kcal, or 100 kcal of the human milk cream composition per day. In some embodiments, the subject is administered about or at least 10 kcal of the human milk cream composition per day. In certain embodiments, the subject is administered about or at least 25 kcal of human milk cream composition per day. In some embodiments, the subject is administered from 1 kcal to 100 kcal, 5 kcal to 75 kcal, 10 kcal to 50 kcal, 10 kcal to 100 kcal, 25 kcal to 75 kcal, 50 kcal to 100 kcal, 25 kcal to 60 kcal, or 25 kcal to 50 kcal of the human milk cream composition per day. In certain embodiments, the subject is administered from 5 kcal to about 60 kcal of the human milk cream composition per day. In some embodiments, the subject is administered from 25 to 50 kcal of the human milk cream composition per day. In certain embodiments, the subject is administered from 25 to 60 kcal of the human milk cream composition per day.

In some embodiments, a dosage, e.g., an oral dosage, of the human milk cream composition is, is about, or is at least 1 kcal, 2 kcal, 2.5 kcal, 3 kcal, 4 kcal, 5 kcal, 6 kcal, 7 kcal, 7.5 kcal, 8 kcal, 9 kcal, 10 kcal, 12.5 kcal, 15 kcal, 17.5 kcal, 20 kcal, 25 kcal, 30 kcal, 35 kcal, 40 kcal, 45 kcal, 50 kcal, 55 kcal, 60 kcal, 62.5 kcal, 65 kcal, 70 kcal, 75 kcal, 80 kcal, 85 kcal, 90 kcal, 95 kcal, or 100 kcal per dose. In particular embodiments, the dosage, e.g., oral dosage, of the human milk cream composition is from 1 kcal to 100 kcal, 5 kcal to 75 kcal, 10 kcal to 50 kcal, 25 kcal to 75 kcal, or 25 kcal to 50 kcal per dose. In certain embodiments, the dosage, e.g., oral dosage, of the human milk cream composition is from 2.5 kcal to about 50 kcal per dose. In some embodiments, the dosage, e.g., oral dosage, of the human milk cream composition is from 20 to 30 kcal per dose. In particular embodiments, the dosage, e.g., oral dosage, is or is about 25 kcal per dose.

In certain embodiments, the subject is administered, e.g., orally, a volume of, of about, or of at least 0.5 mL, 1 mL, 2 mL, 3 mL, 4 mL, 5 mL, 6 mL, 7 mL, 8 mL, 9 mL, 10 mL, 12 mL, 14 mL, 15 mL, 16 mL, 18 mL, 20 mL, 25 mL, 30 mL, 35 mL, 40 mL, 45 mL, 50 mL, 60 mL, 70 mL, 75 mL, 80 mL, 90 mL, or 100 mL of the human milk cream composition per day. In certain embodiments, the subject is administered a volume of from 0.5 mL to 50 mL, 1 mL to 25 mL, 5 mL to 30 mL, or 10 mL to 20 mL of the human milk cream composition per day. In some embodiments, the subject is administered a volume of from 1 mL to 100 mL, 25 mL to 75 mL, 5 mL to 50 mL, 10 mL to 40 mL, or 30 mL to 60 mL of the human milk cream composition per day. In particular embodiments, the volume is from 5 mL to 30 mL per day. In some embodiments, the volume is from 5 mL to 100 mL per day. In certain embodiments, the volume is from 10 mL to 25 mL per day. In some embodiments, the volume is from 10 mL to 20 mL per day.

In some embodiments, a dosage, e.g., an oral dosage, of the human milk cream composition is, is about, or is at least 0.5 mL, 1 mL, 2 mL, 3 mL, 4 mL, 5 mL, 6 mL, 7 mL, 8 mL, 9 mL, 10 mL, 12 mL, 14 mL, 15 mL, 16 mL, 18 mL, 20 mL, or 25 mL per dose. In particular embodiments, the dosage, e.g., oral dosage, of the human cream composition is from 0.5 mL to 25 mL, 1 mL to 10 mL, 5 mL to 15 mL, 7.5 mL to 12.5 mL, or 5 mL to 20 mL per dose. In certain embodiments, the dosage, e.g., oral dosage, of the human cream composition is from 0.5 mL to 25 mL per dose. In some embodiments, the dosage, e.g., oral dosage, of the human milk cream composition is from 5 mL to 10 mL per dose. In particular embodiments, the dosage, e.g., oral dosage, of the human milk cream composition is from 7.5 mL and 12.5 mL per dose.

In certain embodiments, a 10 mL dose of the human milk cream composition is administered, e.g., orally, to the subject, at least once per day. In particular embodiments, a 10 mL dose is administered, e.g., orally, to the subject at least twice per day. In some embodiments, a dose of about 25 kcal of the human milk cream composition is administered, e.g., orally, to the subject at least once per day. In certain embodiments, a dose of about 25 kcal of the human milk cream composition is administered, e.g., orally, to the subject at least twice per day.

In some embodiments, the human milk cream composition is administered to a subject, e.g., an infant, having or suspected of having failure to thrive. In certain embodiments, 1 mL to 25 mL of the human milk cream composition is administered, e.g., orally administered, daily to a subject, e.g., an infant, at least until one or more of the subject's weight, weight for length, or BMI for age is above the fifth percentile. In certain embodiments, 1 mL to 25 mL of the human milk cream composition is administered, e.g., orally administered, daily to a subject, e.g., an infant, at least until one or more of the subject's weight, weight for length, or BMI for age is above the tenth percentile. In particular embodiments, 5 kcal to 75 kcal of the human milk cream composition is administered, e.g., orally administered, daily to a subject, e.g., an infant, at least until one or more of the subject's weight, weight for length, or BMI for age is above the fifth percentile. In some embodiments, 5 kcal to 75 kcal of the human milk cream composition is administered, e.g., orally administered, daily to a subject, e.g., an infant, at least until one or more of the subject's weight, weight for length, or BMI for age is above the tenth percentile.

In some embodiments, 1 mL to 25 mL of the human milk cream composition is administered, e.g., orally administered, daily to a subject, e.g., an infant, at least until the subject increases by at least one major growth percentile for weight for age or for weight for length/height. In particular embodiments, 1 mL to 25 mL of the human milk cream composition is administered, e.g., orally administered, daily to a subject, e.g., an infant, at least until the subject increases by at least two major growth percentiles for weight for age or for weight for length/height. In various embodiments, 1 mL to 25 mL of the human milk cream composition is administered, e.g., orally administered, daily to a subject, e.g., an infant, at least until the subject's percentile for weight for age or weight for length/height is at least the same as the subject's percentile at birth.

In particular embodiments, the human milk cream composition is administered to a subject, e.g., an infant, having or suspected of having failure to thrive. In certain embodiments, 10 mL to 20 mL of the human milk cream composition is administered, e.g., orally administered, daily to a subject, e.g., an infant, at least until one or more of the subject's weight, weight for length, or BMI for age is or increases to above the tenth percentile. In particular embodiments, 25 kcal to 60 kcal of the human milk cream composition is administered, e.g., orally administered, daily to a subject, e.g., an infant, at least until one or more of the subject's weight, weight for length, or BMI for age is or increases to above the fifth percentile. In some embodiments, 25 kcal to 60 kcal of the human milk cream composition is administered, e.g., orally administered, daily to a subject, e.g., an infant, at least until one or more of the subject's weight, weight for length, or BMI for age is or increases to above the tenth percentile. In certain embodiments, 10 mL to 20 mL of the human milk cream composition is administered, e.g., orally administered, daily to a subject, e.g., an infant, at least until the subject increases by at least two major growth percentiles for weight for age or for weight for length/height. In some embodiments, 10 mL to 20 mL of the human milk cream composition is administered, e.g., orally administered, daily to a subject, e.g., an infant, at least until the subject's percentile for weight for age or weight for length/height is or increases to the same as the subject's percentile at birth.

In some embodiments, the human cream composition is fed or administered to a subject to induce weight gain and/or growth velocity in a subject having or suspected of having failure to thrive. In certain embodiments, the human milk cream composition is administered at least once daily, e.g., orally such as via syringe dosing, in an amount of 1 mL to 25 mL per day and/or 5 kcal to 75 kcal per day. In particular embodiments, 10 mL to 20 mL of the human milk cream composition and/or 25 kcal to 60 kcal of the human milk cream composition is administered per day. In particular embodiments, the human milk cream composition is or includes from 20% and 30% human milk fat (weight/volume): from 2.0 and 3.0 kcal/mL: and/or from 75% to 95% of the total calories from human milk fat. In certain embodiments, human milk cream composition is or includes about 24% to 26% human milk fat (weight/volume) and about 2.5 kcal/mL to 2.7 kcal/mL.

In some embodiments, the human cream composition is fed or administered to a subject to induce weight gain and/or growth velocity in a subject. In certain embodiments, the subject's weight for age or weight for length/height has fallen by two major percentiles (e.g., the 95%, 90%, 75%, 50%, 25%, 10%, and 5% percentiles) since birth. In particular embodiments, the human milk cream composition is fed or administered to induce weight gain and/or increase growth velocity in a subject who is below the tenth percentile for (i) sex and corrected age, (ii) weight for length, or (iii) BMI for age. In some embodiments, the human milk cream composition is administered at least once daily until the subject's weight for age or weight for length/height increases by at least two major percentiles and/or increases to the subject's percentile at birth. In certain embodiments, the human milk cream composition is fed or administered at least once daily until the subject's growth percentile is at or above the tenth percentile for (i) sex and corrected age, (ii) weight for length, or (iii) BMI for age. In some embodiments, the human milk cream composition is administered orally, e.g., via syringe dosing, in an amount of 1 mL to 25 mL per day and/or 5 kcal to 75 kcal per day. In particular embodiments, 10 mL to 20 mL of the human milk cream composition and/or 25 kcal to 60 kcal of the human milk cream composition is administered per day. In particular embodiments, the human milk cream composition is or includes from 20% and 30% human milk fat (weight/volume): from 2.0 and 3.0 kcal/mL: and/or from 75% to 95% of the total calories from human milk fat. In certain embodiments, human milk cream composition is or includes about 24% to 26% human milk fat (weight/volume) and about 2.5 kcal/mL to 2.7 kcal/mL.

### II. Human Milk Cream Compositions

Provided herein are human milk cream compositions that are useful, inter alia, for increasing growth velocity or weight gain in a subject in need thereof, e.g., an infant having or suspected of having failure to thrive, or for treating or preventing failure to thrive in a subject in need thereof, e.g., an infant. In certain embodiments, the provided human milk cream compositions have a relatively high fat and low carbohydrate content. In some embodiments, the provided human milk cream compositions have a relatively high fat and low protein content.

In certain embodiments, the human milk cream composition is or includes human milk fat. In certain embodiments, the human milk cream composition contains at least 10%, 15%, 20%, 25%, 30%, 35%, or 40% human milk fat, e.g., by weight of the cream over total volume (w/v) or by weight of the cream over total weight (w/w). In certain embodiments, the human milk cream composition contains at least 3%, 5%, 10%, 15%, 20%, 25%, 30%, 35%, or 40% human milk lipids, e.g., w/w or w/v. In particular embodiments, the human cream composition is or includes from 10% to 40%, from 18% to 30%, from 20% to 30%, from 22.5% to 27.5%, or from 24% to 26% human milk fat, e.g., w/w or w/v. In some embodiments, the human cream composition is or includes from 10% to 40%, from 18% to 30%, from 20% to 30%, from 22.5% to 27.5%, or from 24% to 26% human milk lipids, e.g., w/w or w/v.

In particular embodiments, the human milk cream composition contains at least 100 mg/ mL, 125 mg/mL, 150 mg/mL, 200 mg/mL, 225 mg/mL, 250 mg/mL, 275 mg/mL, 300 mg/mL, 325 mg/mL, 350 mg/mL, 375 mg/mL, 400 mg/mL human milk fat. In some embodiments, the human milk cream composition contains at least 100 mg/ mL, 125 mg/mL, 150 mg/mL, 200 mg/mL, 225 mg/mL, 250 mg/mL, 275 mg/mL, 300 mg/mL, 325 mg/mL, 350 mg/mL, 375 mg/mL, 400 mg/mL human milk lipids. In certain embodiments, the human cream composition is or includes from 100 mg/mL to 400 mg/mL, from 150 mg/mL to 350 mg/mL, 175 mg/mL to 300 mg/mL, 180 mg/mL to 300 mg/mL, 200 mg/mL and 300 mg/mL, 225 mg/mL and 275 mg/mL, or 240 mg/mL to 260 mg/mL human milk fat. In some embodiments, the human cream composition is or includes from 100 mg/mL to 400 mg/mL, from 150 mg/ml to 350 mg/mL, 175 mg/mL to 300 mg/mL, 180 mg/mL to 300 mg/mL, 200 mg/mL and 300 mg/mL. 225 mg/mL and 275 mg/mL, or 240) mg/mL to 260 mg/mL human milk lipids.

In some embodiments, the human milk cream composition contains or includes at least 70%, 75%, 80%, 81%, 82%, 83%, 84%, 85%, 86%, 87%, 88%, 89%, or 90% of the total calories from human milk fat. In certain embodiments, the calories from human milk fat make up at least 70%, 75%, 80%, 81%, 82%, 83%, 84%, 85%, 86%, 87%, 88%, 89%, or 90% of the total calories of the human milk cream composition. In particular embodiments, the human milk cream composition contains or includes from 70% to 95%, 80% to 90%, 85% to 95%, or 85% to 90% of the total calories from human milk fat. In certain embodiments, the calories from human milk fat make up from 70% to 95%, 80% to 90%, 85% to 95%, or 85% to 90% of the total calories of the human milk cream composition. In particular embodiments, about or at least 87% of the total calories of the human milk cream composition are from human milk fat. In certain embodiments, about or at least 88% of the total calories of the human milk cream composition are from human milk fat.

In various embodiments, the human milk cream composition contains or includes at least 70%, 75%, 80%, 81%, 82%, 83%, 84%, 85%, 86%, 87%, 88%, 89%, or 90% of the total calories from human milk lipids. In certain embodiments, the calories from human milk lipids make up at least 70%, 75%, 80%, 81%, 82%, 83%, 84%, 85%, 86%, 87%, 88%, 89%, or 90% of the total calories of the human milk cream composition. In particular embodiments, the human milk cream composition contains or includes from 70% to 95%, 80% to 90%, 85% to 95%, or 85% to 90% of the total calories from human milk lipids. In some embodiments, the calories from human milk lipids make up from 70% to 95%, 80% to 90%, 85% to 95%, or 85% to 90% of the total calories of the human milk cream composition. In some embodiments, about or at least 87% of the total calories of the human milk cream composition are from human milk lipids. In certain embodiments, about or at least 88% of the total calories of the human milk cream composition are from human milk lipids.

In some embodiments, the human milk cream composition is or includes from 18% to 30% human milk fat, e.g., weight/volume (w/v). In certain embodiments, the human milk cream composition is or includes from 18% to 30% human milk lipids e.g., w/v. In certain embodiments, the human milk cream composition is or includes from 20% to 30% human milk fat, e.g., w/v. In certain embodiments, the human milk cream composition is or includes from 20% to 30% human milk lipids, e.g., w/v. In some embodiments, the human milk cream composition is or is about 25% human milk fat, e.g., w/v. In particular embodiments, the human milk cream composition is or is about 25% human milk lipids, e.g., w/v.

In certain embodiments, the human milk cream composition is free or substantially free from non-human milk proteins, e.g., proteins that are not found in or native to human milk. Thus, in particular embodiments, the human milk cream composition does not contain any proteins from any non-human milk, e.g., cow's milk, or any plant or synthetic proteins. In some embodiments, the human cream composition is free or substantially free from non-human milk lipids, e.g., lipids that are not found in or native to human milk.

In certain embodiments, the human milk cream composition includes no more than 15 mg/mL, 12 mg/mL, or 10 mg/mL human milk protein. In particular embodiments, the human milk cream composition includes less than 10 mg/mL human milk protein. In certain embodiments, the human milk cream composition includes less than 100 mg/mL, 90 mg/mL, 80 mg/mL, or 70 mg/mL human milk carbohydrates. In certain embodiments, the human milk cream composition is free or essentially free of non-human milk macronutrients (e.g., fats, lipids, proteins, and carbohydrates). In particular embodiments, the human milk cream composition is free or is essentially free of fats, lipids, proteins, and carbohydrates that are not derived from, found in, or native to human milk.

In particular embodiments, the human milk cream composition is treated to reduce bioburden. In some embodiments, the human milk cream composition is pasteurized. In certain embodiments, the human milk cream composition is a pasteurized human milk cream composition.

In some embodiments, the human milk cream composition is or includes about or at least 0.1 kcal/mL, 0.5 kcal/mL, 1.0 kcal/mL, 1.5 kcal/mL, 2.0 kcal/mL, 2.5 kcal/mL, 2.57 kcal/mL, 3 kcal/mL, or 3.5 kcal/mL. In certain embodiments, the human milk cream composition is or includes from 1.0 kcal/mL to 3.0 kcal/mL, from 2.0 kcal/mL to 3.0 kcal/mL, from 2.25 kcal/mL to 2.75 kcal/mL, or from 2.5 kcal/mL to 2.6 kcal/mL. In some embodiments, the human milk cream composition is or is about 2.5 kcal/mL. In particular embodiments, the human milk cream composition is or is from 2.5 kcal/mL to 2.6 kcal/mL. In certain embodiments, the human milk cream composition is or is about 2.57 kcal/mL.

In certain embodiments, the human milk cream composition is from 18% to 30% human milk fat or human milk lipids, e.g., w/v, and from 2.0 kcal/mL to 3.0 kcal/mL. In certain embodiments, the human milk cream composition is from 20% to 30% human milk fat or human milk lipids, e.g., w/v, and from 2.0 kcal/mL to 3.0 kcal/mL. In some embodiments, the human milk cream composition is from 22.5% to 27.5% human milk fat, e.g., w/v, or human milk lipids and from 2.25 kcal/mL to 2.75 kcal/mL. In certain embodiments, the human milk cream composition is from 24% to 26% human milk fat, e.g., w/v, or human milk lipids and from 2.5 kcal/mL to 2.6 kcal/mL. In certain embodiments, the human milk cream composition is or is about 25% human milk fat, e.g., w/v, and is or is about 2.5 kcal/mL. In particular embodiments, the human milk cream composition is or is about 25% human milk fat, e.g., w/v, and is or is about 2.57 kcal/mL.

In some embodiments, the human milk cream composition is a human milk composition described in or is a human milk cream composition made by a process described in U.S. Patent Application No.: 2014/0272027.

### III. Manufacture of Human Milk Cream Compositions

The human milk cream compositions described herein are produced from whole human milk. The human milk may be obtained from an infant's own mother or from one or more donors. In certain embodiments, the human milk is pooled to provide a pool of human milk. In some embodiments, the human milk is pooled from the milk of at least 2, 5, 10, 25, 50, 100, 150, or 200 or more different individual human milk donors.

### A. Obtaining Donor Milk

In certain embodiments, human milk donors are pre-screened and approved before any milk is processed. Various routine and known techniques are used to identify and qualify suitable donors. A potential donor must obtain a release from her physician and her child's pediatrician as part of the approval process. This helps to insure, inter alia, that the donor is not chronically ill and that her child will not suffer as a result of the donation(s). Methods and systems for qualifying and monitoring milk collection and distribution are described, e.g., in U.S. patent application Ser. No. 12/728,811 (U.S. 2010/0268658), which is incorporated herein by reference in its entirety. Donors may or may not be compensated for their donation.

In some embodiments, donor screening includes a comprehensive lifestyle and medical history questionnaire that includes an evaluation of prescription and non-prescription medications, testing for drugs of abuse, and testing for certain pathogens. In certain embodiments, the donor may be screened for some or all of human immunodeficiency virus Type 1 (HIV-1), HIV-2, human T-lymphotropic virus Type 1 (HTLV-I), HTLV-II, hepatitis B virus (HBV), hepatitis C virus (HCV), COVID-19, and syphilis. In some embodiments, positive test results for drugs or abuse or pathogens disqualify the individual from milk donation.

In some aspects, donors may be periodically requalified. For example, a donor is required to undergo screening by the protocol used in their initial qualification every four months if the donor wishes to continue to donate. A donor who does not requalify or fails qualification is deferred until such time as they do, or permanently deferred if warranted by the results of requalification screening. In the event of the latter situation, all remaining milk provided by that donor is removed from inventory and destroyed or used for research purposes only.

A donor may donate at a designated facility (e.g., a milk bank office) or, in a preferred embodiment, express milk at home. If the donor will be expressing milk at home, she will measure the temperature in her freezer with, e.g., a supplied thermometer to confirm that it is cold enough to store human milk in order to be approved.

Once the donor has been approved, donor identity matching may be performed on donated human milk because the milk may be expressed by a donor at her home and not collected at a milk banking facility. In a particular embodiment, each donor's milk can be sampled for genetic markers, e.g., DNA markers, to guarantee that the milk is truly from the approved donor. Such subject identification techniques are known in the art (see, e.g., International Application Serial No. PCT/US2006/36827, which is incorporated herein by reference in its entirety). The milk may be stored (e.g., at −20° C. or colder) and quarantined until the test results are received.

For example, the methods featured herein may include a step for obtaining a biological reference sample from a potential human breast milk donor. Such sample may be obtained by methods known in the art such as, but not limited to, a cheek swab sample of cells, or a drawn blood sample, milk, saliva, hair roots, or other convenient tissue. Samples of reference donor nucleic acids (e.g., genomic DNA) can be isolated from any convenient biological sample including, but not limited to, milk, saliva, buccal cells, hair roots, blood, and any other suitable cell or tissue sample with intact interphase nuclei or metaphase cells. The sample is labeled with a unique reference number. The sample can be analyzed at or around the time of obtaining the sample for one or more markers that can identify the potential donor. Results of the analysis can be stored, e.g., on a computer-readable medium. Alternatively, or in addition, the sample can be stored and analyzed for identifying markers at a later time.

It is contemplated that the biological reference sample may be DNA typed by methods known in the art such as STR analysis of STR loci, HLA analysis of HLA loci or multiple gene analysis of individual genes/alleles. The DNA-type profile of the reference sample is recorded and stored, e.g., on a computer-readable medium.

It is further contemplated that the biological reference sample may be tested for self-antigens using antibodies known in the art or other methods to determine a self-antigen profile. The antigen (or another peptide) profile can be recorded and stored, e.g., on a computer-readable medium.

A test sample of human milk is taken for identification of one or more identity markers. The sample of the donated human milk is analyzed for the same marker or markers as the donor's reference sample. The marker profiles of the reference biological sample and of the donated milk are compared. The match between the markers (and lack of any additional unmatched markers) would indicate that the donated milk comes from the same individual as the one who donated the reference sample. Lack of a match (or presence of additional unmatched markers) would indicate that the donated milk either comes from a non-tested donor or has been contaminated with fluid from a non-tested donor.

The donated human milk sample and the donated reference biological sample can be tested for more than one marker. For example, each sample can be tested for multiple DNA markers and/or peptide markers. Both samples, however, need to be tested for at least some of the same markers in order to compare the markers from each sample.

Thus, the reference sample and the donated human milk sample may be tested for the presence of differing identity marker profiles. If there are no identity marker profiles other than the identity marker profile from the expected subject, it generally indicates that there was no fluid (e.g., milk) from other humans or animals contaminating the donated human milk. If there are signals other than the expected signal for that subject, the results are indicative of contamination. Such contamination will result in the milk failing the testing.

The testing of the reference sample and of the donated human milk can be carried out at the donation facility and/or milk processing facility. The results of the reference sample tests can be stored and compared against any future donations by the same donor.

The milk is then tested for pathogens. The milk may be genetically screened, e.g., by polymerase chain reaction (PCR), to identify, e.g., viruses, such as including but not limited to HIV-1 and 2, HTLV-1 and 2, HBV, HCV, and COVID-19, or bacteria such as syphilis. A microorganism panel that screens for various bacterial species, fungus and mold via culture may also be used to detect contaminants. For example, a microorganism panel may test for aerobic count, Bacillus cereus, Escherichia coli, Salmonella, Pseudomonas, coliforms, Staphylococcus aureus, yeast, and mold. In particular, B. cereus is a pathogenic bacterium that cannot be removed through pasteurization. Pathogen screening may be performed both before and after pasteurization.

In addition to screening for pathogens, the donor milk may also be tested for drugs of abuse (e.g., cocaine, opiates, synthetic opioids (e.g. oxycodone/oxymorphone) methamphetamines, benzodiazepine, amphetamines, and THC) and/or adulterants such as non-human proteins. For example, an ELISA may be used to test the milk for a non-human protein, such as bovine proteins, to ensure, e.g., that cow milk or cow milk infant formula has not been added to the human milk, for example to increase donation volume when donors are compensated for donations.

### B. Processing Human Milk

Once the human milk has been screened, it is processed to produce a high fat product, e.g., a human cream composition. The donation facility and milk processing facility can be the same or different facility. Processing of milk can be carried out with large volumes of human milk, e.g., about 75 liters/lot to about 2,000 liters/lot of starting material.

After the human milk is carefully analyzed for both identification purposes and to avoid contamination as described above, the milk then undergoes filtering, e.g., through about a 200 micron filter, and heat treatment. For example, the composition can be treated at about 63° C. or greater for about 30 minutes or more.

In certain embodiments, the cream, e.g., human milk cream, is obtained from a method or technique that is or includes creaming separation or centrifugation. In some aspects, creaming separation allows for the combination of fat globules over time thus allowing for the accumulation of fat globules in a cream layer towards the surface of the milk. In some aspects, whole milk may be heated, e.g., to between 20° C. and 50° C. (68° F. and 122° F.), to increase the speed of the fat globule combinations and accumulation in a cream layer. In particular embodiments, the topical formulation is or includes cream, e.g., human milk cream, obtained or collected by creaming separation. In some embodiments, the cream is obtained by centrifugation of whole milk. In certain aspects, centrifugation relies on a spinning whole milk in a centrifuge, e.g., at between 5,000 and 10,000 RCF, to produce cream and skim layers. In certain aspects, the cream fraction may be further centrifuged, e.g., such as to enrich or concentrate the lipid or fatty acid content. In certain embodiments, the topical formulation contains cream, e.g., human milk cream, that is separated or obtained by centrifugation.

In particular embodiments, the human milk cream contains only human milk components, e.g., lipids, fatty acids, fats, carbohydrates, sugars, oligosaccharides, or proteins that are concentrated, isolated, or derived from, native to, or found in human milk. In particular embodiments, the human milk cream is free or essentially free of non-human lipids, fatty acids, fats, carbohydrates, sugars, oligosaccharides, or proteins, e.g., lipids, fatty acids, fats, carbohydrates, sugars, oligosaccharides, or proteins that are not concentrated, isolated, or derived from, native to, or found in human milk. In certain embodiments, the human milk cream is free or essentially free of xenogeneic components or compounds, such as including but not limited to lipids, fatty acids, fats, carbohydrates, sugars, oligosaccharides, or proteins.

Following the separation of cream and skim, the skim portion undergoes further filtration, e.g., ultrafiltration. This process concentrates the nutrients in the skim milk by filtering out the water. This process results in a permeate fraction (that passes through the ultra-filter) and a retentate fraction (that does not pass through filter). The resulting retentate and skim portions may be further processed to produce human milk fortifiers (e.g., PROLACTPLUS™ Human Milk Fortifiers, e.g., PROLACT+4™, PROLACT+6™ PROLACT+8™, and/or PROLACT+10™, which are produced from human milk and contain various concentrations of nutritional components) and the compositions of the fortifiers are described in U.S. patent application Ser. No. 11/947,580, filed on Nov. 29, 2007, (U.S. 2008/0124430) the contents of which are incorporated herein in their entirety. These fortifiers can be added to the milk of a nursing mother to enhance the nutritional content of the milk for, e.g., a preterm infant.

Once the human milk cream portion has been separated from the human skim milk portion, the caloric content of the human milk cream portion is measured. In certain embodiments, a volume of deionized water, distilled water, and/or the permeate from the ultrafiltration of the skim milk is added to the human milk cream portion, e.g., to achieve a target fat, lipid, and/or calorie content. In some embodiments, deionized water, distilled water, and/or permeate is added to human cream to achieve a human milk cream composition having a target human milk fat or lipid content from 18% to 30% (e.g., w/w or w/v) and a target calorie content of from 2.0 kcal/mL to 3.0 kcal/mL. In certain embodiments, deionized water, distilled water, and/or permeate is added to human cream to achieve a human milk cream composition having a target human milk fat or lipid content from 20% to 30% (e.g., w/w or w/v) and a target calorie content of from 2.0 kcal/mL to 3.0 kcal/mL. In some embodiments, deionized water, distilled water, and/or permeate is added to human cream to achieve a human milk cream composition having a target human milk fat or lipid content from 22.5% to 27.5% (e.g., w/w; or w/v) and a target calorie content of from 2.25 kcal/mL to 2.75 kcal/mL. In particular embodiments, deionized water, distilled water, and/or permeate is added to human cream to achieve a human milk cream composition having a target human milk fat or lipid content from 24% to 26% (e.g., w/w or w/v) and a target calorie content of from 2.5 kcal/mL to 2.6 kcal/mL. In certain embodiments, deionized water, distilled water, and/or permeate is added to human cream to achieve a human milk cream composition having a target human milk fat or lipid content of or of about 25% (e.g., w/w or w/v) and a target calorie content of or of about 2.5 kcal/mL. In some embodiments, deionized water, distilled water, and/or permeate is added to human cream to achieve a human milk cream composition having a target human milk fat or lipid content of or of about 25% (e.g., w/w or w/v) and a target calorie content of or of about 2.57 kcal/mL.

After the human milk cream has been processed to achieve target fat, lipid, and/or calorie contents, the resulting human milk cream composition undergoes one or more steps to reduce bioburden. Processes that reduce bioburden are known and include, but are not limited to, pasteurization. In some embodiments, pasteurization is high-temperature, short-time pasteurization (HTST). In certain embodiments, the composition can be placed in a process tank that is connected to the high-temperature, short-time (HTST) pasteurizer via platinum-cured silastic tubing. After pasteurization, the cream composition can be collected into a second process tank and cooled. In some embodiments, the pasteurization is vat pasteurization. In some embodiments, the human milk cream composition is heated to a minimum of 63° C. and held at that temperature for a minimum of thirty minutes. The air above the cream composition is steam heated to at least three degrees Celsius above the cream composition temperature. In particular embodiments, the temperature of the human milk cream composition is about 66° C. or greater, the air temperature above the cream composition is about 69° C. or greater, and these temperatures held for or for about 30 minutes or longer. In certain embodiments, both HTST and vat pasteurization are performed.

In certain embodiments, after the human milk cream composition has been treated to reduce bioburden it is generally processed aseptically. In certain embodiments, the human milk cream composition is cooled, e.g., to or to about 2 to 8° C., after the treatment to reduce bioburden, e.g., pasteurization. In particular embodiments, the cooled, pasteurized human milk cream composition is filled into filled into containers of desired volumes. In particular embodiments, various samples of the composition are taken for nutritional and bioburden analysis. The nutritional analysis ensures proper calorie and fat content of the cream composition. In particular embodiments, a label that reflects the nutritional analysis is generated for each container. In some embodiments, the bioburden analysis tests for presence of contaminants, e.g., total aerobic count, B. cereus, E. coli, Coliform, Pseudomonas, Salmonella, Staphylococcus, yeast, and/or mold. The bioburden testing may be genetic testing. The product is packaged and shipped once the analysis is complete and desired results are obtained.

In one embodiment, the resultant human cream composition comprises about 2.0 kcal to about 3.0 kcal or more per ml. In a preferred embodiment, the human cream composition comprises about 2.5 kcal/ml. It is contemplated that the resultant human cream composition comprises about 20% to about 30% or more fat, e.g., w/v. In one embodiment, the human cream composition is about 25% fat, e.g., w/v. In certain embodiments, the resultant human cream composition is a pasteurized human cream composition containing from 18% to 30% or 20% to 30%, optionally about 25%, human milk fat (w/v) and from 2.0 kcal/mL to 3.0 kcal/mL or 2.5 kcal/mL to 2.7 kcal/mL.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless specified otherwise, concentrations of a given substance in a mixture or solution that are expressed as percentages refers to the percent as calculated by the weight of the given substance in grams (g) over the volume of the mixture or solution per 100 milliliters (mL). For example, a 5% concentration of substance X in solution Y refers to a concentration of 5 g of substance X per 100 mL of solution Y.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the described or claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some aspects, “about” a value means within a range of +25%, +10%, +5%, +1%, +0.1%, or +0.01% of the value.

By “milk” is meant the fluid that is produced by the mammary gland of a mammal and expressed by the breast. Milk includes all lactation products including, but not limited to colostrum, whole milk and skim milk taken at any point post parturition or during the cycle of lactation, in the production of hind milk and foremilk. Unless otherwise specified, as used herein “milk” refers typically to whole human milk.

Unless otherwise indicated, as used herein the term “percentile” or “growth percentile” refers to the number for which a given value for sex and corrected age, weight for length or height, or BMI for age where a certain percentage of scores fall below that number. Particular embodiments contemplate that an infant's percentile for sex and corrected age, weight for length or height, or BMI for age can be determined as a matter of routine, such as by consulting the World Health Organization's (WHO's) charts length/height-for-age, weight-for-age, weight-for-length, weight-for-height, and body mass index-for-age or those provided by the CDC.

By “substantially” as in “substantially free of”' a substance, e.g., xenogeneic compounds, it is meant that the substance is completely absent or present in negligible concentrations or amounts that cannot be detected by standard or routine techniques and/or are present at concentrations below those that could be considered biologically active.

By “consisting essentially” of, as used herein refers to compositions containing particular recited components while excluding other major bioactive factors.

### EXAMPLES

The following examples are included for illustrative purposes only and are not intended to limit the scope of the invention.

### Example 1

### Manufacture of a Human Milk Cream Composition

Human milk from previously screened and approved donors was mixed together to generate a pool of donor milk. In a clean room environment, the pool of donor milk was further tested for specific pathogens and bovine proteins. Specifically, PCR testing was used to screen for the presence of pathogens including HIV-1 and 2, HTLV 1 and 2, HBV, HCV, Zika, syphilis, and COVID-19 in the milk. A microbiological panel was also performed that tests for Bacillus cereus, Escherichia coli, Salmonella, Pseudomonas, coliforms, Staphylococcus aureus, yeast, and mold.

The pool of donor milk was ultra-centrifuged to generate a cream portion and a skim milk portion. The cream portion was then formulated to meet specific fat and calorie specifications by adding an amount of the permeate ultra-filtered from the skim portion. Specifically, the cream portion was standardized to 25% lipids and contained about 2.5 kcal/ml.

The standardized cream composition was then pasteurized (approximately 63° C. (145º F) for 30 minutes) following guidance set by the FDA's Pasteurized Milk Ordinance. Following pasteurization, the standardized cream composition was then filled into high density polyethylene bottles and frozen. The bottles were weighed to ensure that the intended volume was filled into the bottle. The bottled cream composition was then quarantined until all data from the microbiological panel was reviewed and a full nutritional analysis was performed.

### Example 2

### Treatment of Failure to Thrive with a Human Milk Cream Composition

A two-month-old breastfed female infant was falling off her respective growth trajectory for an undetermined reason. The infant was given supplemental feedings with 10 ml of a human milk cream composition produced similar to as described in Example 1 once per day for two weeks via syringe dosing in addition to continued breastfeeding. An increased growth velocity was observed in the infant during the two weeks of the feedings with the human milk cream composition.

### Example 3

### Improving Growth Velocity with a Human Milk Cream Composition

A breastfed female infant with a congenital lung malformation was supplemented with feedings of cow-based formula to increase weight ahead of corrective surgery. The formula feedings were not tolerated by the infant, and so the supplemental formula feedings were replaced by twice daily oral feedings of a human milk cream composition produced similar to as described in Example 1 provided approximately an extra 53 kcal/day. The supplemental feedings with the human milk cream composition began when the infant was 4.5 months old and weighed 11 lb. 2.5 oz. (5.06 kg). Eight days into the treatment, the infant had gained 5 oz. (142 g), and at twelve days infant had gained 11 oz. (312 g).

Treatment will continue until just prior to the surgery. If the infant achieves adequate growth (returns to the percentile for weight for age she had at birth), supplemental feedings with the human milk cream composition may be adjusted or reduced to allow for the infant to maintain her growth trajectory.

1. A method of treating failure to thrive in a subject, the method comprising administering to a subject in need thereof a human milk cream composition, wherein the human milk cream composition comprises from 20% to 30% human milk fat and from 2.0 kcal/mL to 3.0 kcal/mL.

2. The method of claim 1, wherein the human milk cream composition is free or essentially free of macronutrients of a non-human milk origin.

3. The method of claim 1 or 2, wherein the human milk cream composition is administered orally.

4. The method of claim 1 or 2, wherein the human milk cream composition is administered enterally.

5. The method of any of claims 1-4, wherein the human milk cream composition is not mixed or diluted with one or more compositions prior to administering.

6. The method of any of claims 1-4, wherein the human milk cream composition is mixed with human milk prior to administering.

7. The method of any of claims 1-6, wherein the human milk cream composition is administered to the subject in a dose of from 1 mL to 20 mL.

8. The method of any of claims 1-7, wherein the subject is administered at least one dose of the human milk cream composition per day.

9. The method of any of claims 1-8, wherein the subject is administered from 5 mL to 100 mL of the human milk cream composition per day.

10. The method of any of claims 1-9, wherein the human milk cream composition is administered to the subject in an amount sufficient for the subject to achieve a growth rate of at least 20 g/day.

11. The method of any of claims 1-10, wherein the human milk cream composition is administered to the subject in an amount sufficient for the subject to achieve a growth rate of 20 g/day to 30 g/day.

12. The method of any of claims 1-11, wherein the subject is less than two years old

13. The method of any of claims 1-12, wherein the subject is breastfed and/or receives an exclusive human milk diet.

14. The method of any of claims 1-13, wherein the subject is a term infant, a late preterm infant, and/or was at least 34 weeks gestation at birth.

15. The method of any of claims 1-14, wherein the failure to thrive is associated with or suspected of being associated with inadequate calorie intake, inadequate nutrient absorption, or increased metabolism.

16. The method of any of claims 1-15, wherein the failure to thrive is associated with or suspected of being associated with inadequate calorie intake, wherein the inadequate calorie intake is associated with one or more of gastroesophageal reflux, inadequate breast milk supply or ineffective latching, mechanical feeding difficulties, poor oral neuromotor coordination, toxin-induced or infectious gastrointestinal upset.

17. The method of any of claims 1-16, wherein the failure to thrive is associated with or suspected of being associated with inadequate nutrient absorption, wherein the inadequate nutrient absorption is associated with one or more of anemia or iron deficiency, biliary atresia, celiac disease, food intolerance, food allergy, or chronic gastrointestinal conditions.

18. The method of any of claims 1-17, wherein the failure to thrive is associated with or suspected of being associated with increased metabolism, wherein the increased metabolism is associated with a chronic infection, chronic lung disease of prematurity, congenital or acquired cardiovascular disease, hyperthyroidism, inflammatory conditions, malignancy, congenital adrenal overactivity, hypertension, a hormone secreting tumor, or renal dysfunction.

19. The method of any of claims 1-18, wherein the subject has or is suspected of having congenital anomalies, developmental delay, gastroesophageal reflux, low birth weight, or poor oral health.

20. The method of any of claims 1-19, wherein the subject has
(i) weight below the 10th percentile for sex and corrected age:
(ii) weight for length below the 10th percentile:
(iii) body mass index for age below the 10th percentile: and/or
(iv) a decrease in growth velocity, whereby the subject's weight for age or weight for length/height has fallen by two major percentiles since birth, wherein the major percentiles are 95%, 90%, 75%, 50%, 25%, 10%, and 5%.

21. The method of any of claims 1-20, wherein the human milk cream composition has been treated with a treatment to reduce bioburden.

22. The method of claim 21, wherein the treatment to reduce bioburden comprises pasteurization.

23. The method of any claims 1-22, wherein the human milk cream composition comprises cream from human milk of multiple donors.

24. The method of claim 23, wherein the cream is from human milk of at least 5, 10, 25, 50, or 100 individual donors.

25. The method of any of claims 1-24, wherein the human milk cream composition comprises from 24% to 26% human milk fat and from 2.25 kcal/mL to 2.75 kcal/mL.

26. The method of any of claims of 1-25, wherein the human milk cream composition comprises about 25% human milk fat and 2.5 kcal/mL to 2.7 kcal/mL.

27. A use of a human milk cream composition for treating failure to thrive in a subject in need thereof, wherein the human milk cream composition comprises from 20% to 30% human milk fat and from 2.0 to 3.0 kcal/mL.

28. Human milk cream composition for use in the manufacture of a medicament for the treatment of failure to thrive in a subject in need thereof, wherein the human milk cream composition comprises from 20% to 30% human milk fat and from 2.0 to 3.0 kcal/mL.

29. A method for inducing weight gain in a subject in need thereof, wherein the subject's weight for age or weight for length/height has fallen by two major percentiles since birth, the method comprising enterally or orally administering to the subject in need thereof a human milk cream composition comprising from 20% and 30% human milk fat and from 2.0 and 3.0 kcal/mL:
wherein the subject is less than 2 years old, was at least 34 weeks gestational age at birth, and is breastfed or exclusively breastfed,
wherein the human milk cream composition is administered at least once daily for a period of time sufficient for the subject's weight for age or weight for length/height to increase by two major percentiles,
wherein the major percentiles are 95%, 90%, 75%, 50%, 25%, 10%, and 5%.

30. A method for inducing weight gain in a subject in need thereof, wherein the subject's weight for age, weight for length/height, or body mass index for age is below the tenth percentile, the method comprising enterally or orally administering to the subject in need thereof a human milk cream composition comprising from 20% and 30% human milk fat and from 2.0 and 3.0 kcal/mL:
wherein the subject is less than 2 years old, was at least 34 weeks gestational age at birth, and is breastfed or exclusively breastfed,
wherein the human milk cream composition is administered at least once daily for a period of time sufficient for the subject's weight for age, weight for length/height, or body mass index for age to increase to at least the tenth percentile.

31. The method of claim 29 or 30, wherein the human milk fat composition is administered orally.

32. The method of any of claims 29-31, wherein from 10 kcal to 100 kcal of the human milk cream composition is administered daily.

33. The method of any of claims 29-32, wherein the human milk cream composition comprises about 25% human milk fat and 2.5 kcal/mL to 2.7 kcal/mL.
